(19) United States
(12) Patent Application Publication
(10) Pub. No.: US 2025/0262345 A1
(43) Pub. Date: Aug. 21, 2025

(54) COMPOSITES AND DEVICES FOR INTERFACING ELECTRONICS TO BIOLOGICAL TISSUE

(71) Applicant: The Trustees of Columbia University in the City of New York, New York, NY (US)

(72) Inventors: Dion Khodagholy, Mission Viejo, CA (US); Patricia Jastrzebska-Perfect, New York, NY (US)

(21) Appl. No.: 19/029,843

(22) Filed: Jan. 17, 2025

Related U.S. Application Data
(63) Continuation of application No. 17/022,004, filed on Sep. 15, 2020, now Pat. No. 12,201,741.
(60) Provisional application No. 62/900,633, filed on Sep. 15, 2019.

(51) Int. Cl. A61L 24/00; A61B 5/25; A61B 5/259; A61L 24/08; C08G 61/12; C08L 5/00; C08L 5/08; C08L 81/02; C09D 165/00; C09J 105/00

(52) U.S. Cl. CPC A61L 24/0094; A61B 5/259; A61L 24/001; A61L 24/08; C08G 61/126; C08L 5/00; C08L 5/08; C08L 81/02; C09D 165/00; C09J 105/00; A61B 5/25; A61B 2562/0217; C08G 2261/11; C08G 2261/1424; C08G 2261/3223; C08G 2261/794; C08L 2203/02

(57) ABSTRACT

Composites, are provided, the composites comprising: mixed conducting particles; and an ion conducting scaffolding matrix. In some embodiments, the mixed conducting particles are made from poly(3,4-ethylenedioxythiophene)-poly(styrenesulfonate). In some embodiments, the ion conducting scaffolding matrix includes a chitosan (CS)-based polymer. In some embodiments, devices are provided, the devices comprising: a composite comprising mixed conducting particles and an ion conducting scaffolding matrix; and three electrodes, wherein: each of the three electrodes is in contact with the composite; a first pair of the three electrodes are on opposite sides of the composite and are a distance h apart; a second pair of the three electrodes are on a same side of the composite and are a distance d1 apart; a particle size of the mixed conducting particles is between h and d1; a mean-free-path of the mixed conducting particles is less than d1; and the composite behaves like an anisotropic conductor.

### CROSS REFERENCE TO RELATED APPLICATION

This application is a continuation of U.S. patent application Ser. No. 17/022,004, filed Sep. 15, 2020, which claims the benefit of U.S. Provisional Patent Application No. 62/900,633, filed Sep. 15, 2019, each of which is hereby incorporated by reference herein in its entirety.

### BACKGROUND

For a variety of reasons, it is desirable to interface electronics to biological tissue. For example, it is desirable to be able to interface electronics to people and animals for the purposes of diagnostics and medical treatment.

Current devices for interfacing electronics to biological tissue are inadequate.

For example, high spatiotemporal resolution conformable probes are increasingly being used to acquire signals from biological tissues, and similar resolution rigid electronics exist to process these signals. However, current techniques to enable transmission of signals between the soft probe and hard electronics (e.g., thermal bonding or sonic metal-metal bonding, metal/epoxy composite pastes) are not biocompatible or scalable, and introduce additional rigidity and bulk.

As another example, conventional electrodes designed to acquire electrophysiologic signals from the skin typically rely on ionic gels to form an appropriate impedance interface with the skin. These gels are not amenable to patterning and their spread across the skin surface is difficult to control, limiting the spatial resolution of activity that can be acquired non-invasively.

Accordingly, new mechanisms for interfacing electronics to biological tissue are desirable.

### SUMMARY

In accordance with some embodiments, mechanisms (which can include composites, methods, and devices) for interfacing electronics to biological tissue are provided. In some embodiments, composites, are provided, the composites comprising: mixed conducting particles; and an ion conducting scaffolding matrix. In some of these embodiments, the mixed conducting particles are made from poly(3,4-ethylenedioxythiophene)-poly(styrenesulfonate). In some of these embodiments, the ion conducting scaffolding matrix includes a chitosan (CS)-based polymer.

In some embodiments, devices are provided, the devices comprising: a composite comprising mixed conducting particles and an ion conducting scaffolding matrix; and three electrodes, wherein: each of the three electrodes is in contact with the composite; a first pair of the three electrodes are on opposite sides of the composite and are a distance h apart; a second pair of the three electrodes are on a same side of the composite and are a distance d1 apart; a particle size of the mixed conducting particles is between h and d1; a mean-free-path of the mixed conducting particles is less than d1; and the composite behaves like an anisotropic conductor.

In some embodiments, devices are provided, the devices comprising: a composite comprising mixed conducting particles and an ion conducting scaffolding matrix; and three electrodes, wherein: each of the three electrodes is in contact with the composite; the three electrodes are on a same side of the composite; a first pair of the three electrodes are a distance d1 apart; a second pair of the three electrodes are a distance d2 apart, where d2 is greater than d1; a particle size of the mixed conducting particles is between d1 and d2; and the composite behaves like an ionic transistor.

In some embodiments, devices are provided, the devices comprising: a composite comprising mixed conducting particles and an ion conducting scaffolding matrix; and three electrodes, wherein: each of the three electrodes is in contact with the composite; the three electrodes are on a same side of the composite; a first pair of the three electrodes are a distance d1 apart; a second pair of the three electrodes are a distance d2 apart, where d2 is greater than d1; and the composite behaves like a resistor, and wherein at least one of: a particle size of the mixed conducting particles is greater than d2; and a mean-free-path of the mixed conducting particles is less than d1.

In some embodiments, devices are provided, the devices comprising: a composite comprising mixed conducting particles and an ion conducting scaffolding matrix; and three electrodes, wherein: each of the three electrodes is in contact with the composite; the three electrodes are on a same side of the composite; a first pair of the three electrodes are a distance d1 apart; a second pair of the three electrodes are a distance d2 apart, where d2 is greater than d1; a particle size of the mixed conducting particles is between d1 and d2; a mean-free-path of the mixed conducting particles is between d1 and d2; and the composite behaves like an independently gated ionic transistor.

In some embodiments, devices are provided, the devices comprising: a composite comprising mixed conducting particles and an ion conducting scaffolding matrix; and three electrodes, wherein: each of the three electrodes is in contact with the composite; the three electrodes are on a same side of the composite; a first pair of the three electrodes are a distance d1 apart; a second pair of the three electrodes are a distance d2 apart, where d2 is greater than d1; a particle size of the mixed conducting particles is less than d1; a mean-free-path of the mixed conducting particles is between d1 and d2; and the composite behaves like a diode.

### BRIEF DESCRIPTION OF THE DRAWINGS

FIG. 1A1 is an example illustration of a diameter (α) and a mean-free-path (λ) of mixed conducting particles in an ionic space in accordance with some embodiments.

FIG. 1A2 is an example of a schematic of a particulate mixed-conducting composite placed in between a substrate top layer having electrodes and a substrate bottom layer having electrodes in accordance with some embodiments.

FIG. 1A3 is an example graph showing that particulate mixed-conducting composites can provide different functional modes of operation depending on the combination of the particle size (diameter)(α) and the mean free path (λ) of mixed-conducting particles in the composites in accordance with some embodiments.

FIG. 1B is an example illustration of a process for forming mixed-conducting particles in accordance with some embodiments.

FIGS. 1C1-1C4 are example illustrations of sieving mixed-conducting particles in accordance with some embodiments.

FIG. 2A is an example of a graph showing homogeneity of a distribution of mixed-conducting particles in low density and high density particulate mixed conducting composites in accordance with some embodiments.

FIG. 2B is an example of a graph showing distributions of different particles sizes of mixed-conducting particles after four different sieving processes in accordance with some embodiments.

FIG. 2C is an example of a schematic showing a composite sandwiched between two substrates that include multiple horizontally spaced, vertically aligned strips of gold in accordance with some embodiments.

FIG. 2D is an example of graph showing different resistances measured for different composites in accordance with some embodiments.

FIG. 3A is an example of another schematic showing a composite sandwiched between two substrates that include multiple horizontally spaced, vertically aligned strips of gold in accordance with some embodiments.

FIG. 3B is an example of a graph showing different resistances measured in composites in accordance with some embodiments.

FIG. 3C is an example of a graph showing different resistances measured in composites over time in accordance with some embodiments.

FIG. 3D is an example of a schematic of a particulate mixed conducting composite-based independently gated ionic transistor in accordance with some embodiments.

FIG. 3E is an example of graph showing characteristics of a particulate mixed conducting composite-based independently gated ionic transistor in accordance with some embodiments.

FIG. 3F is an example of a NOR gate constructed using a composite in accordance with some embodiments.

FIG. 3G is an example of a graph showing the behavior of a NOR gate constructed using a composite in accordance with some embodiments.

FIG. 3H is an example of a diode constructed using a composite in accordance with some embodiments.

FIG. 3I is an example of a graph showing the behavior of a diode constructed using a composite in accordance with some embodiments.

FIG. 4A is an example of a graph showing high gamma oscillations across electrodes of a particulate mixed conducting composite-bonded array placed on a cortical surface of a freely moving rat in accordance with some embodiments.

FIG. 4B is an example of a graph showing that trigger-averaged gamma band power can be spatially confined across a particulate mixed conducting composite-bonded array placed on a cortical surface of a freely moving rat in accordance with some embodiments.

FIG. 4C is an example of a graph showing high fidelity acquisition of characteristic high frequency hippocampal oscillations measured using penetrating probes in accordance with some embodiments.

FIG. 4D is an example of a graph showing burst firing of a putative pyramidal cell zoomed in from the location denoted by the white star in FIG. 4C in accordance with some embodiments.

FIG. 4E is an example of a graph showing sample wide-band local field potential (LFP) traces (0.1-1250 Hz) acquired during intra-operative human recording demonstrating spatially diverse activity patterns acquired by a particulate mixed conducting composite-bonded neural probe in accordance with some embodiments.

FIG. 4F is an example of a spectrogram of neural data acquired by a particulate mixed conducting composite-bonded neural probe revealing a transition from an anesthetized state to an awake state intra-operatively in accordance with some embodiments.

FIG. 4G is an example of a schematic showing gold-based electrodes coated with a layer of a particulate mixed conducting composite applied to a person's skin in accordance with some embodiments.

FIG. 4H is an example of sample traces of a particulate mixed conducting composite-acquired EMG (top) and ECG (bottom) measured in accordance with some embodiments.

FIG. 4I is an example of five graphs showing differentiable patterns of neural activity across an electrode array during voluntary flexion of each finger on a hand measured in accordance with some embodiments.

FIG. 4J is an example of a graph showing that multiple adjacent electrodes on an array captured the same action potential in accordance with some embodiments.

FIG. 4K is an example of a current source density heat map of a sample nerve action potential from FIG. 4J as visualized across adjacent electrodes in accordance with some embodiments.

### DETAILED DESCRIPTION

In accordance with some embodiments, mechanisms, which can include composites, methods, and devices, for interfacing electronics to biological tissue are provided. More particularly, in some embodiments, particulate mixed-conducting composites for interfacing electronics to biological tissue are provided.

In some embodiments, these particulate mixed-conducting composites include mixed-conducting particles in an ion conducting scaffolding polymer matrix that enable electronics to be interfaced to biological tissue. In some embodiments, this is accomplished by controlling the sparsity and the size of the mixed-conducting particles within a scaffolding polymer matrix that has controllable ionic conductivity.

In some embodiments, the particulate mixed-conducting composites allows a single material to function as multiple different principle electronic components. In some embodiments, the particulate mixed-conducting composites also eliminate the need for several bonded layers of patterned conducting, semiconducting, and insulating materials in order to provide multiple different principle electronic components.

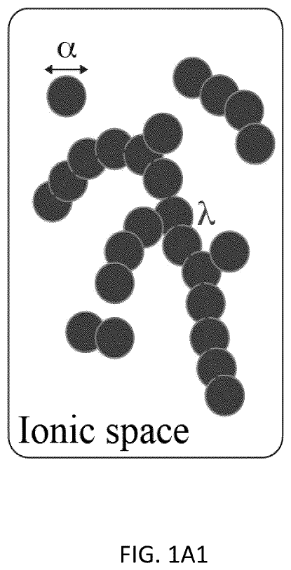

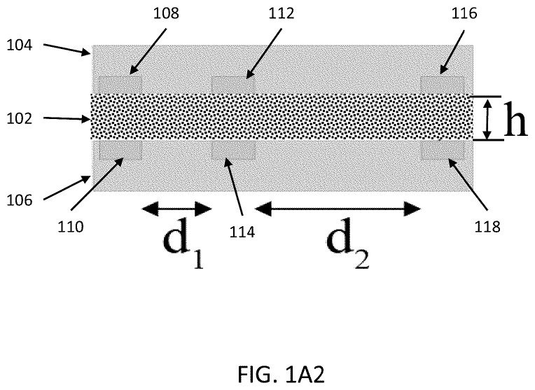

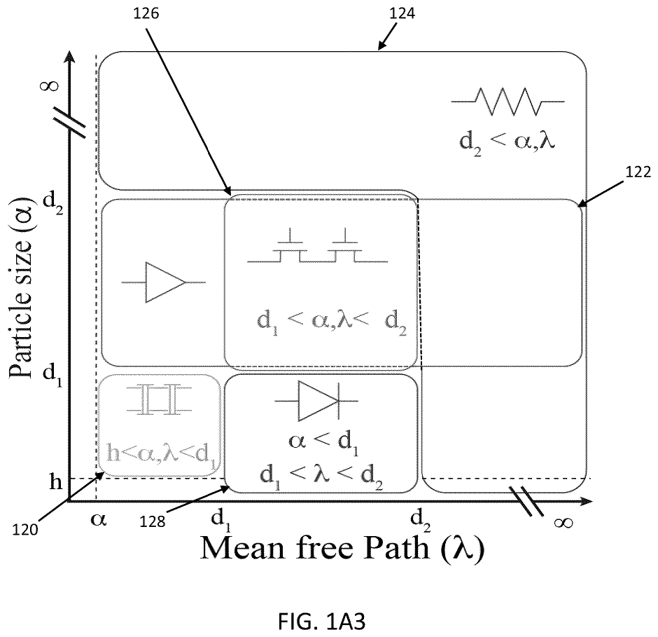

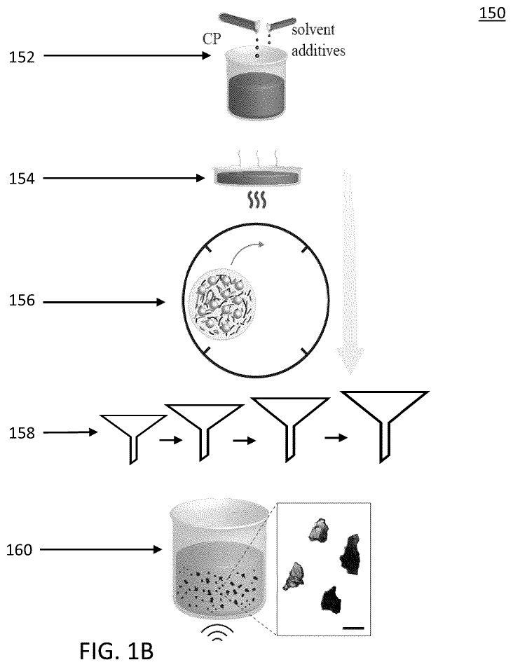

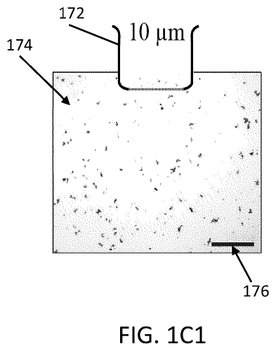

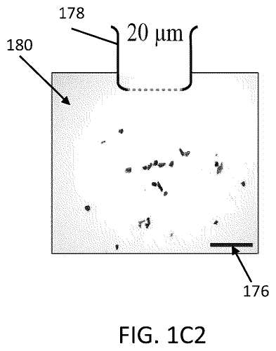

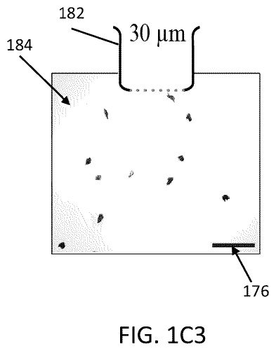

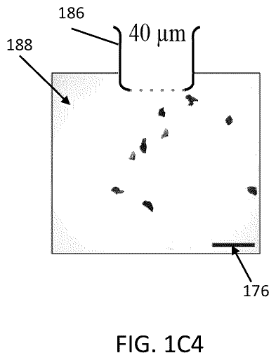

In some embodiments, key properties of the particulate mixed-conducting composites are the mean diameter (α) of the mixed-conducting particles and the density (ρ) of the mixed-conducting particles in the ion conducting scaffolding polymer matrix. The mean diameter α and density ρ interact to determine the longest electrically conducting length that the particles can form (mean-free-path, λ). In some embodiments, the mean-free-path λ is proportional to mean diameter α multiplied by the density ρ. An example illustration of diameter (α) and mean-free-path (λ) in accordance with some embodiments is shown in FIG. 1A1

In some embodiments, as shown in FIG. 1A2, the particulate mixed-conducting composites 102 can be placed in between a substrate top layer 104 having electrodes 108, 112, and 116 and a substrate bottom layer 106 having electrodes 110, 114, and 118. As also shown, in some embodiments, the distance between the electrodes in top layer 104 and the electrodes in bottom layer 106 can be defined as distance h, the shortest distance between adjacent electrodes (e.g., the distances between electrodes 108 and 112 and between electrodes 110 and 114) can be defined as distance d₁, and the longest distance between electrodes (e.g., the distances between electrodes 112 and 116 and between electrodes 114 and 118) can be defined as distance d₂.

In some embodiments, as shown in FIG. 1A3 by regions 120, 122, 124, 126, and 128, the particulate mixed-conducting composites can provide different functional modes of operation depending on the combination of the particle size (diameter)(α) and the mean free path (λ) of the mixed-conducting particles relative to distances d₁, d₂, and h.

More particularly for example, as shown by region 120, a particulate mixed-conducting composite can behave as an anisotropic conductor in some embodiments. This can occur when the particle size α and the density ρ of the particles result in a mean free path λ that is shorter than the distance d₁ and the particle size α is between distance h and distance d₁. This is the case in some embodiments because: when the mean free path λ is shorter than the distance d₁, the possibility of lateral electric conduction is eliminated because no continuous electrical path can be formed by the particles; and when the particle size α is also approximately equal to the distance h, selective vertical conduction will occur.

As another example, as shown by region 122, a particulate mixed-conducting composite can behave as an ionic transistor in some embodiments. This can occur when the particle size α and the density ρ of the particles result in a mean free path λ that is longer than distance d₁ that defines the transistor's channel length. This is the case in some embodiments because, when the particle size is large enough to bridge distance d₁, the particles form a transistor channel that can be doped and/or dedoped by the ionic conducting scaffolding polymer matrix when a voltage is applied.

As still another example, as shown by region 124, a particulate mixed-conducting composite can behave as a resistor in some embodiments. This can occur when the particle size α is greater than the distance d₂ and/or the particle size α and the density ρ of the particles result in a mean free path λ that is longer than the distance d₂. This is the case in some embodiments because when the particle size is orders of magnitude longer than d₁ and/or the mean free path λ is orders of magnitude longer than the distance d₁, resistive electronic properties will dominate the interaction and the composite will approach the properties of a conducting polymer.

As yet another example, as shown by region 126, a particulate mixed-conducting composite can behave as an independently gated ionic transistor in some embodiments. This can occur when the particle size α is between the distance d₁ and the distance d₂ and the mean free path λ is between the distance d₁ and the distance d₂. In this case, the gate to channel distance is defined by distance d₂ while channel length is distance d₁. This is the case in some embodiments because: increasing the particle size α or the density ρ to create a mean free path π that is longer than the distance d₁ but shorter than the distance d₂ allows for independent gating of the ionic transistor; and particles bridging the distance d₁ form a transistor channel, which has ionic interaction with the gate electrode located at the distance d₂ through the scaffolding polymer matrix. In this manner, addressable transistors can be formed without channel patterning in some embodiments.

As still another example, as shown by region 128, a particulate mixed-conducting composite can behave as a diode in some embodiments. This can occur when the particle size α is less than the distance d₁ and the particle size α and the density ρ of the particles result in a mean free path λ that is between the distance d₁ and the distance d₂. This can be the case in some embodiments because when the particle size α is substantially less than the distance d₁, but the mean free path λ is approximately equal to the distance d₁, a diode is created. Particle chains that are in contact with the electrodes and span the distance d₁ (bridging particle chains) permit electronic conduction to occur between the terminals. However, particle chains that are omnidirectionally located within the scaffolding polymer matrix will induce ion conduction and dope and/or dedope the bridging particles, resulting in a nonlinear relationship between applied voltage and conduction.

An example of a process of preparing particulate mixed conducting composites is now provided.

In some embodiments, mixed conducting particles can be formed by example process 150 as shown in FIG. 1B.

As illustrated, beginning at 152, a conducting dispersion of a conducting polymer, including additives and crosslinkers to maximize conductivity and stability, can be formed. Any suitable conducting polymer, additives, and crosslinkers can be used in some embodiments. For example, in some embodiments, a highly conducting dispersion of the conducting polymer poly(3,4-ethylenedioxythiophene)-poly(styrenesulfonate) (PEDOT:PSS) can be used. More particularly, for example, in some embodiments, a high-conductivity PEDOT:PSS can be prepared by mixing 80% PEDOT:PSS, 20% ethylene glycol, and 0.6% DBSA (all v/v). PEDOT:PSS (Clevios™ PH 1000) is available from Heraeus Deutschland GmbH & Co. KG of Leverkusen, Germany. Ethylene glycol, 4-dodecyl benzene sulfonic acid (DBSA), and poly(styrene sulfonate) are available from MilliporeSigma of St. Louis, MO.

Next, at 154, the dispersion can be evaporated over a large surface area to create dried, highly conductive sheets. This dispersion can be evaporated in any suitable manner, and these sheets can have any suitable thickness, such as a thickness of less than 50 μm, in some embodiments. More particularly, for example, in some embodiments, high-conductivity PEDOT:PSS mixed with 1% v/v of 3-glycidoxypropyltrimethoxysilane (GOPS) can be cast into a glass petri dish and dehydrated for eight to sixteen hours at 120° C. (or any other suitable temperature) in a well-ventilated environment. 3-glycidyloxypropyltrimethoxysilane (GOPS) is available from MilliporeSigma of St. Louis, MO.

Then, at 156, the resulting film can be cut into small fragments and crushed in a bead mill. This can be performed in any suitable manner in some embodiments. For example, in some embodiments, the resulting film can be scraped from a petri dish with a stainless-steel blade, cut into small fragments using a blade, suspended in Iso Propyl Alcohol (IPA), and crushed in a bead mill using stainless steel (or any other suitable material) beads having any suitable size (e.g., with diameter of 3.17-6.35 mm) for any suitable period of time (e.g., for eight to sixteen hours).

At 158, the particle suspension can next be serially filtered to produce one or more desired ranges of particle sizes. This filtering can be performed in any suitable manner to produce any one or more ranges of sizes of particles. For example, in some embodiments, the particle suspension can be serially filtered using polyethylene terephthalate mesh (PET-mesh) cell strainers with pores ranging from 1-40 μm in diameter. An illustration of such filtering is shown in FIGS. 1C1, 1C2, 1C3, and 1C4, each having a scale bar 176 of 100 μm in accordance with some embodiments. As shown in FIG. 1C1, the particle suspension can first be filtered using a 10 μm sieve 172 to produce particles 174 having sizes up to 10 μm in some embodiments. As shown in FIG. 1C2, the remaining particle suspension (i.e., what does not pass the sieve in FIG. 1C1) can next be filtered using a 20 μm sieve 178 to produce particles 180 having sizes between 10 μm and 20 μm in some embodiments. As shown in FIG. 1C3, the remaining particle suspension (i.e., what does not pass the sieve in FIG. 1C2) can then be filtered using a 30 μm sieve 182 to produce particles 184 having sizes between 20 μm and 30 μm in some embodiments. As shown in FIG. 1C4, the remaining particle suspension (i.e., what does not pass the sieve in FIG. 1C3) can finally be filtered using a 40 μm sieve 186 to produce particles 188 have sizes between 30 μm and 40 μm in some embodiments. Sieves (cell strainers) (e.g., PET-mesh with pore diameters of 10, 20, 30, and 40 μm) can be obtained from PluriSelect USA™ of El Cajon, CA.

Referring back to FIG. 1B, next, at 160, particles in the desired size range(s) can be resuspended in IPA, the suspension can be sonicated, the suspension can be allowed to precipitate after sonication, and excess IPA can be removed after precipitation. The scale bar 160 of FIG. 1B represents 50 μm. Sonication can be performed in any suitable manner and for any suitable duration in some embodiments. For example, in some embodiments, sonication can be performed at 40 kHz for eight to sixteen hours. In some embodiments, further sieving can be performed after sonification to obtain one or more specific particle size(s).

Chitosan (CS)-based polymers can be used to create an ion-conducting scaffolding polymer matrix in some embodiments. In some embodiments, CS is beneficial for this purpose because its ion conductivity (water-uptake) and adhesiveness can be tuned. In some embodiments, CS-based polymers can be used to create an ion-conducting scaffolding polymer matrix in any suitable manner. For example, in some embodiments, CS can be dissolved in 2% v/v acetic acid, filtered using 40 μm cell strainers, and then concentrated via dehydration to 2.5% w/v. A 40% w/v D-sorbitol solution can be prepared with the chitosan stock. PEDOT particles can then be added dropwise into CS-sorbitol under constant stirring to produce a 2.5:1 (unless otherwise specified) PEDOT:CS by weight suspension. The suspension can then be dehydrated to 0.1% w/v of PEDOT in final solution. CS (50-190 kD, 75-85% deacetylated) and D-sorbitol (BioUltra≥99.5%) is available from MilliporeSigma of St. Louis, MO.

The mixed-conducting particles can be combined with the scaffolding polymer matrix to produce the particulate mixed conducting composites. The mixed-conducting particles can be combined with the scaffolding polymer matrix in any suitable manner, such as by mixing them together.

In some embodiments, the particulate mixed conducting composites provide a reliable, flexible mechanical bonding between conformable substrates due to the bio-adhesive properties of chitosan.

In some embodiments, a distinct color contrast between mixed-conducting particles (dark blue) and the scaffolding polymer matrix (light yellow) enables direct optical imaging. In some embodiments, the autofluorescence of CS can additionally or alternatively leveraged for visualization.

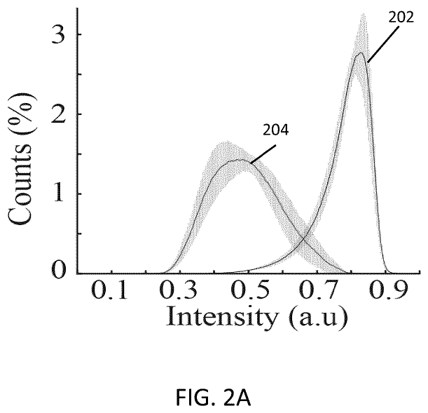

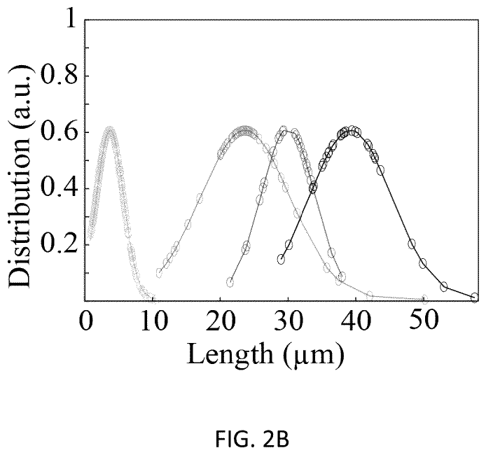

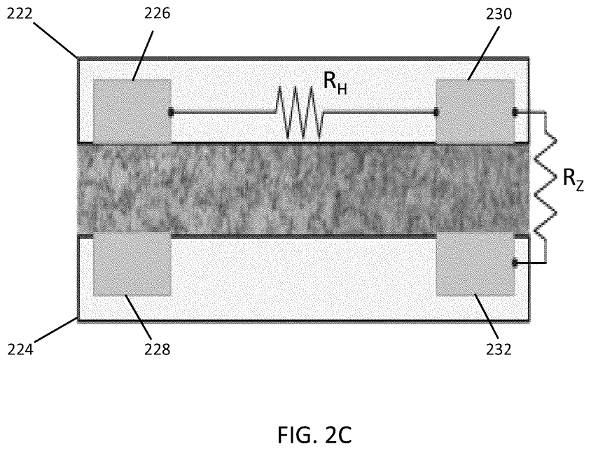

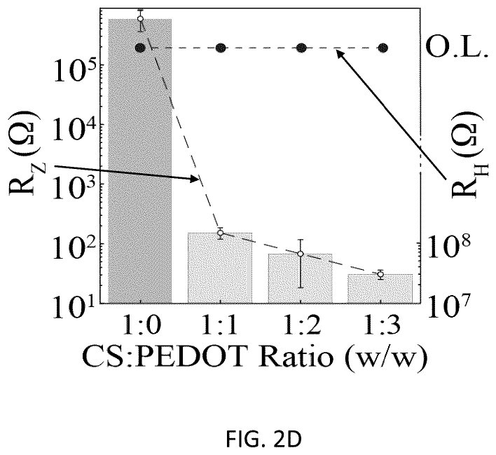

To evaluate the homogeneity of the distribution of the mixed-conducting particles in the particulate mixed conducting composites in some embodiments, composites with a low density (ρ) of mixed-conducting particles and a high density (ρ) of mixed-conducting particles were evaluated to produce the graph of FIG. 2A. Optical analysis of same-sized areas blade-coated with each of a low-density composite and a high-density composite revealed that the majority of the pixels in the low-density composite reflected an absence of particles as shown by curve 202 of FIG. 2A, while the distribution of pixel intensity of the high density composite was shifted to the left (darker), indicating the presence of numerous particles, as shown by curve 204 of FIG. 2A. These results suggest that increasing the density of particles results in a homogeneous composite that is uniformly darker when visualized. If particles instead tended to coagulate into focal areas, a multi-peaked distribution would occur as particle density was increased.

Using similar optical methods, it was found that the sieving process described in connection with FIGS. 1C1, 1C2, 1C3, and 1C4 can be highly selective, creating particles ranging from 10-40 μm with narrow size distributions as shown in FIG. 2B.

To characterize the conductivity of the particulate mixed conducting composites in some embodiments, composites with various particle densities, including one without particles (control), were prepared. As shown in FIG. 2C, these composites were sandwiched between two substrates 222 and 224 that included multiple horizontally spaced, vertically aligned strips of gold (Au) (1 mm wide and 100 μm spacing, 100 nm thick) 226, 228, 230, and 232. As shown in FIG. 2D, the composites (bars with 1:1, 1:2, and 1:3; 1:0 is control with no particles) exhibited less than 100 Ω electrical contact resistance between the vertically aligned Au strips. This vertical resistance (R_{Z}) was inversely proportional to particle density, whereas resistance between horizontally spaced Au strips remained high (RH>10¹⁰ Ω).

These features suggest that the particles remain homogenously distributed within the particulate mixed conducting composites when sandwiched between the substrates, without focal coagulation that would cause increased horizontal conduction in some embodiments. Thus, the physical processes used to prepare the particulate mixed conducting composites offers several key advantages in some embodiments: (i) highly controllable generation of particles with specific size and density; (ii) homogeneous distribution of particles within the composite, even after lamination; (iii) preservation of electrical properties of the conducting polymer; (iv) ability to include additives that enhance electrical performance of the composite; (v) solvent-free synthesis, enhancing compatibility with organic materials; and (vi) scalability for a variety of production volumes.

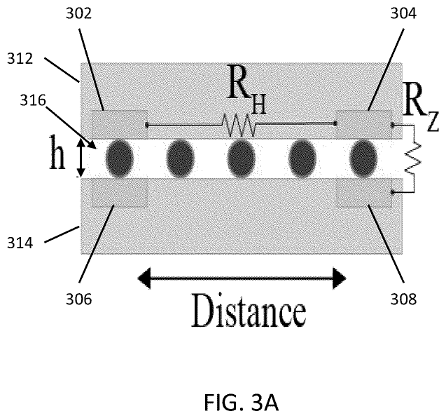

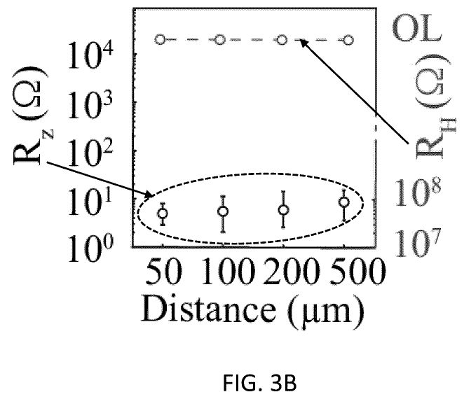

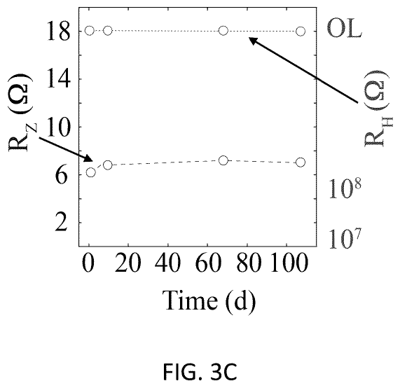

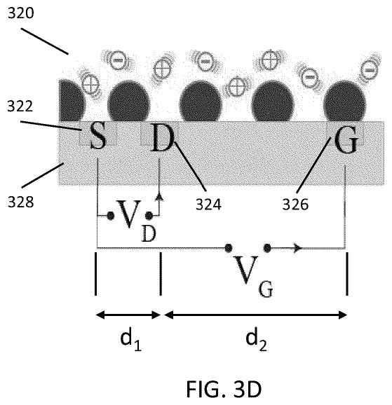

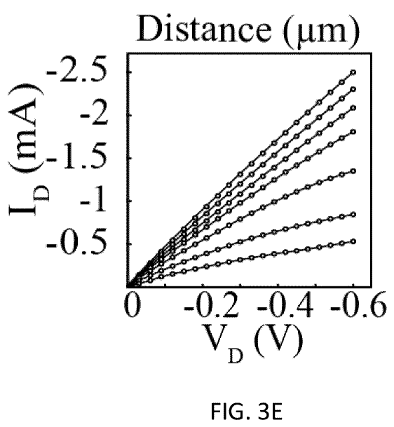

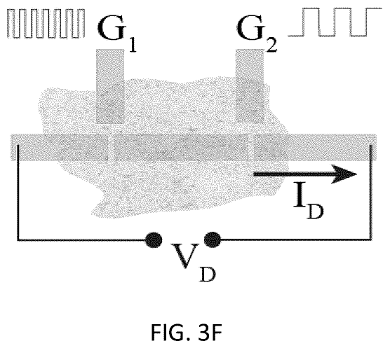

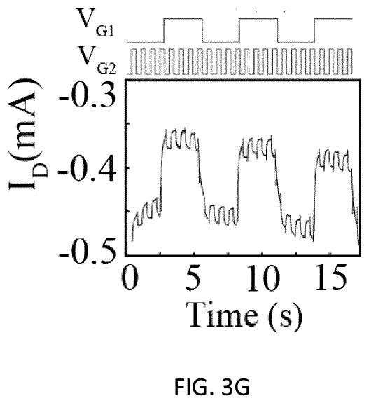

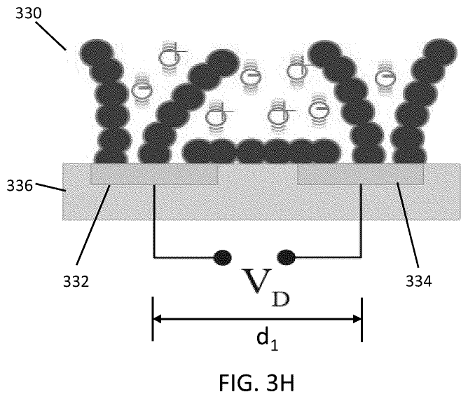

To determine whether a particulate mixed conducting composite has sufficient anisotropy to form high-density vertical interconnects between electronic components without patterning in some embodiments, Au-based electronic pads 302, 304, 306, and 308 were fabricated on rigid (SiO₂) and conformable (parylene C) substrates 312 and 314 with geometrically varying inter-electrode spacing as shown in FIG. 3A. A particulate mixed conducting composite 316 was deposited onto bottom substrate 314 using typical solution processible techniques (e.g., spin coating and blade coating) and then covered by top substrate 312. As described above in connection with FIG. 1A3, particulate mixed conducting composite 316 had α and λ less than Distance in FIG. 3A but greater than h in FIG. 3A. Because CS is intrinsically adhesive, the particulate mixed conducting composite established strong mechanical bonds between layers of both rigid and conformable substrates, without requiring elevated temperatures (maximum 70° C.) or application of pressure. Anisotropy (A) of the interface was determined by calculating the ratio (A=R_{H}/R_{Z}) of the horizontal resistance (R_{H}) between pads 302 and 304 and pads 306 and 308, and vertical resistance (R_{Z}) between pads 302 and 306 and pads 304 and 308. Ensuring that the particulate mixed conducting composite used had a smaller λ than the electrode distance, we were able to achieve anisotropy values of at least 10⁶ as shown in FIG. 3B, and as high as 10¹⁰, at 50 μm resolution. As shown in FIG. 3C for particle size of 40 μm and an electrode pitch of 250 μm, these anisotropy values were consistent for more than 100 days, highlighting the stability of particulate mixed conducting composite's electrical and mechanical bonding.

As shown in FIG. 3D, particulate mixed conducting composite-based independently gated ionic transistors were fabricated by blade-coating particulate mixed conducting composite 320 onto three Au-based pads 322, 324, and 326 (representing a source, a drain, and a gate, respectively) embedded in a substrate 328. As described above in connection with FIG. 1A3, particulate mixed conducting composite 320 had both α and λ between d₁ and d₂. This configuration functioned as a depletion-mode, high-transconductance, individually addressable organic electrochemical transistor. Application of positive gate voltage (V_{G}) across Au pads 322 and 326 directed cations of the ion-conducting scaffolding polymer matrix into the bridging mixed-conducting particles of the channel, resulting in dedoping. The electrical characteristics of the transistor are shown in FIG. 3E. The independent gating of these devices has been used to create logic circuits with series and parallel connectivity between transistor terminals. For example, a NOR gate, as shown in FIG. 3F, has been implemented in which G₁ and G₂ are the inputs. As shown in FIG. 3G, this particulate mixed conducting composite-based transistors performed the appropriate digital logic. Therefore, in some embodiments, particulate mixed conducting composite can be used to produce independent transistors that maintain the properties of their constituent conducting polymer without requiring any channel patterning.

To determine whether the particulate mixed conducting composite can operate as a diode, as shown in FIG. 3H, particulate mixed conducting composite 330 with appropriate α and ρ was coated onto two Au-based pads 332 and 334 embedded in a substrate 336 and a voltage V_{D} was applied between the terminals. As described above in connection with FIG. 1A3, particulate mixed conducting composite 330 had α substantially less than d₁ of FIG. 3H and λ approximately equal to d₁ of FIG. 3H. The application of voltage resulted in both (i) electronic current through the bridging particles of the terminals, and (ii) ionic current between omnidirectional particles. As the voltage increased, cations dedoped the bridging particles and lowered the charge carrier density, generating a non-linear voltage-current relationship characteristic of a diode as shown in FIG. 3I.

In some embodiments, the characteristics of the particulate mixed conducting composites enable high spatiotemporal resolution, biocompatible multi-channel electrical contact between soft and hard electronic device components with a facile fabrication process that is adaptable to a wide range of materials. Conformable (parylene C) and flexible (PEN) high-density neural interface devices for integration with flexible (PET) and rigid (FR4) neural acquisition electronic circuits can be fabricated in some embodiments. For example, in some embodiments, a particulate mixed conducting composite can be applied to the back-end of the probe using a blade-coater. The particulate mixed conducting composite will then form an adhesive film (through evaporation of excess water) (e.g., within 120 s at room temperature). A mating board can then be aligned and a mechanical contact established using a manual cotton-based roller with application of pressure (e.g., 0.15 N).

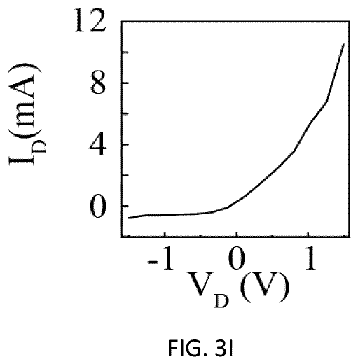

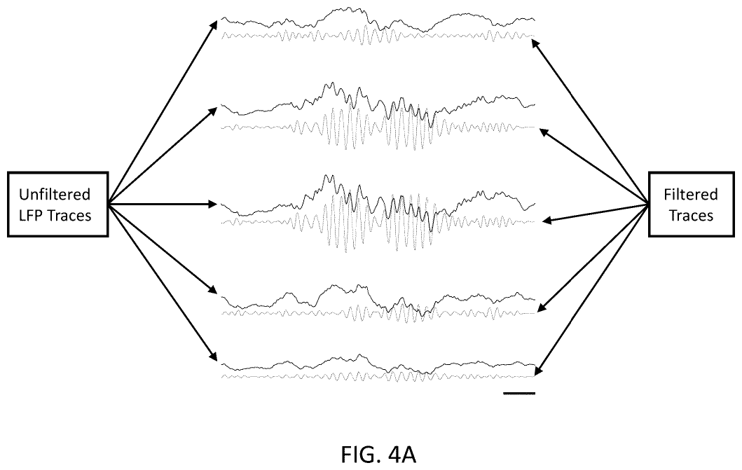

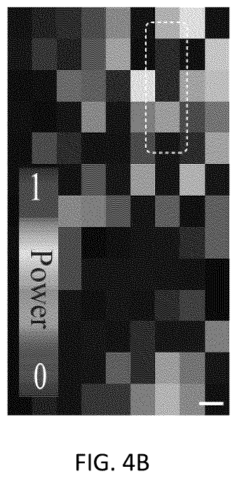

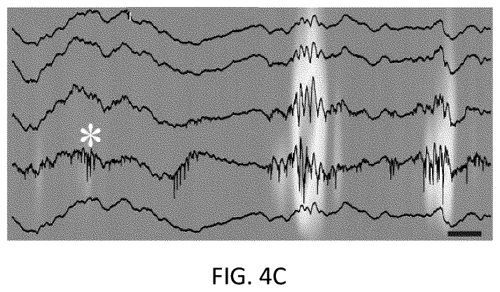

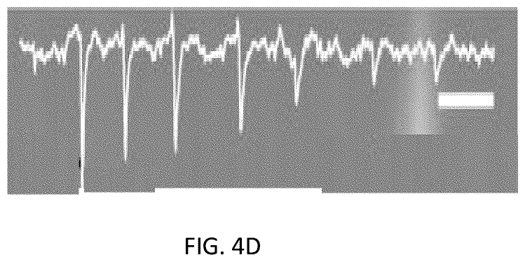

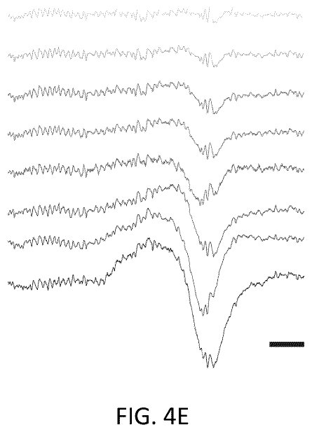

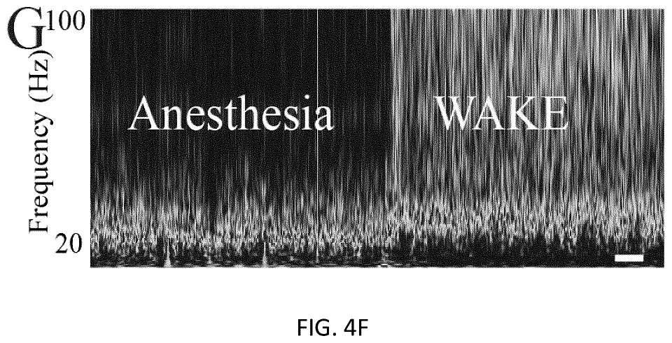

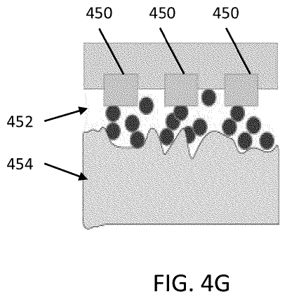

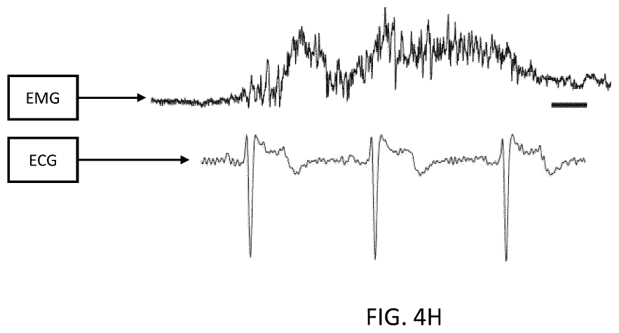

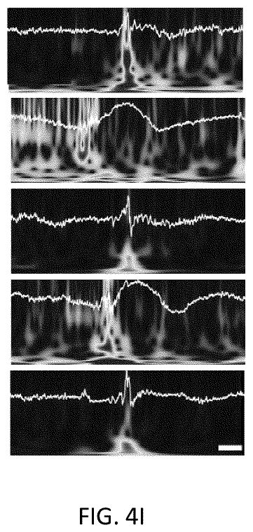

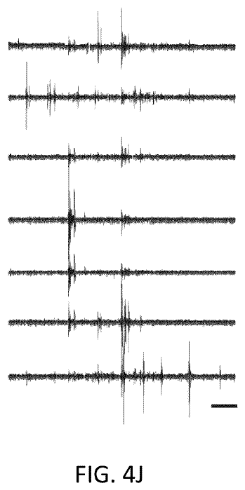

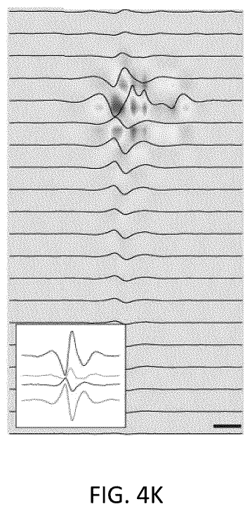

To validate the functionality of such particulate mixed conducting composite-bonded devices, some embodiments were used to acquire high spatiotemporal resolution electrophysiological signals in various experimental conditions. Particulate mixed conducting composite-bonded surface electrocorticography (ECoG) arrays (NeuroGrid; 128 channels) and penetrating probes (32 channels) were implanted chronically into freely moving rodents. NeuroGrid recordings demonstrated spatially localized high gamma oscillations as shown in FIGS. 4A and 4B. FIG. 4A shows that, in some embodiments, high gamma oscillations are differentiable across electrodes of a particulate mixed conducting composite-bonded array placed on a cortical surface of a freely moving rat (unfiltered local field potential (LFP) traces and corresponding filtered traces (60-100 Hz)). The scale bar in FIG. 4A is 40 ms. FIG. 4B shows that, in some embodiments, trigger-averaged gamma band power can be spatially confined across a particulate mixed conducting composite-bonded array placed on a cortical surface of a freely moving rat. The scale bar in FIG. 4B is 1 mm.

Insertion of penetrating probes into the dorsal CA1 of the hippocampus allowed high fidelity acquisition of characteristic high frequency hippocampal oscillations as shown in FIG. 4C (ripples, 100-250 Hz) in accordance with some embodiments. FIG. 4C shows sample wide-band traces (0.1 to 20 KHz) superimposed on a heatmap highlighting the instantaneous power in the ripple band (100-150 Hz). The scale bar in FIG. 4C is 50 ms.

FIG. 4D shows individual action potential waveforms (burst firing of a putative pyramidal cell, zoomed in from the location denoted by the white star of FIG. 4C, 0.1 to 20 KHz). The scale bar in FIG. 4D is 5 ms. Taken together, these results demonstrate that particulate mixed conducting composite bonding can be used to create scalable, multi-channel neural interface devices that are capable of acquiring signals at the spatiotemporal resolution of individual neurons in some embodiments.

The biocompatibility of particulate mixed conducting composites can be used in some embodiments to generate neural interface devices for intra-operative neural recording in human patients undergoing implantation of deep brain stimulation (DBS) electrodes.

In some embodiments, a proximity contact lithography system (Suss MA-6) can be used to pattern Pa-C, Au, Pt, Ti, and PEDOT/PSS films. For example, in some embodiments, first, Pa-C can be deposited using a chemical vapor deposition (SCS Labcoter 2) to a thickness of 2 μm. 3-(trimethoxysilyl) propyl methacrylate can be used as an adhesion promoter and a dilute solution of industrial cleaner (Micro-90) as an anti-adhesion agent. Next, the film can be patterned with a 4.6 μm thick layer of AZ9260 photoresist and dry-etched with a reactive ion etching process (Oxford 80 Plus; 180 W, 50-sccm O2, 3-sccm SF6, 2-sccm CF4 for 18 min). A lift-off process can be used to pattern metal pads and interconnects. AZ nLOF 2020 (chemically amplified negative resist) can be spin-coated on the Pa-C film (5500 rpm), baked at 115° C. for 60 s, exposed using Suss MA-6, and finally developed (AZ developer). With use of an e-beam metal evaporator (Angstrom at 2.10⁻⁶ bars), metallic layers of Ti (10 nm), and Au (150 nm) can be deposited. Lift-off can be performed using 1165 stripper (2 hours). To enhance the conductivity of PEDOT:PSS, a mixture of PEDOT:PSS aqueous dispersion (PH-1000 from H. C. Stark) and ethylene glycol (20:5 ml ratio) can be prepared and mixed with dodecylbenzenesulfonic acid (100 ml per 50 ml) and GOPS (1 w/v to adjust surface energy and cross-link, respectively. The resulting dispersion can be spin-coated in two steps with a soft bake in between (120° C., 60 s) at 650 rpm. The films can be subsequently baked at 140° C. for 1 hour and then immersed in deionized water to remove any excess low-molecular weight compounds. The electrodes can be characterized in vitro using phosphate-buffered saline solution. A significantly larger Au plate compared to electrode geometry can serve as the reference electrode for impedance spectroscopy and measurements.

During a DBS procedure, clinical electrodes can be inserted through a burr hole (which can have any suitable size, e.g., 14 mm diameter) to reach the appropriate subcortical target. In some embodiments, particulate mixed conducting composite-bonded NeuroGrids can be placed within a burr hole and used to acquire spatially resolved, high signal-to-noise-ratio (SNR) neurophysiological data in the intra-operative environment. FIGS. 4E and 4F show data captured in such an embodiment that reveals characteristic signals associated with transition from anesthesia to waking, as well as localized epochs of gamma oscillations. FIG. 4E shows sample wide-band local field potential (LFP) traces (0.1-1250 Hz) acquired during intra-operative human recording demonstrating spatially diverse activity patterns acquired by a particulate mixed conducting composite-bonded neural probe in some embodiments. The scale bar in FIG. 4E is 100 ms. FIG. 4F shows a spectrogram of neural data acquired by a particulate mixed conducting composite-bonded neural probe revealing a transition from an anesthetized state to an awake state intra-operatively. The scale bar in FIG. 4F is 10 s. Thus, particulate mixed conducting composite-bonded neural interface devices can be safely and effectively translated to use in human subjects in some embodiments.

In some embodiments, a particulate mixed conducting composite can be used to interface directly with the human body and enable high spatiotemporal resolution, mechanically stable sensing. For example, as shown in FIG. 4G, in some embodiments, Au-based electrodes 450 coated with a layer of a particulate mixed conducting composite (200 μm diameter) 452 can be applied to a person's skin 454 in some embodiments. More particularly, for example, in some embodiments, electrodes can be placed over a bicep muscle and a wrist of person to acquire EMG and ECG signals. FIG. 4H shows an example of sample traces of a particulate mixed conducting composite-acquired EMG (top) and ECG (bottom) signals measured in accordance with some embodiments. The scale bar in FIG. 4H is 1 s.

To evaluate the possibility of higher spatial resolution biopotential acquisition in some embodiments, a conformable array of electrodes (64 channels, 250 μm electrode diameter, 4×7mm2 effective surface area) was fabricated, a thin layer of particulate mixed conducting composite to a similar surface area of skin over the wrist of a human subject was applied, and the array placed on top. Voluntary flexion of each finger resulted in differentiable patterns of neural activity across the electrode array as shown in the five graphs of FIG. 4I. The scale bar in FIG. 4I is 80 ms. As shown, it was possible to localize independent nerve action potentials with well-defined waveforms and firing rates. As shown in FIG. 4J, multiple adjacent electrodes on the array captured the same action potential with waveforms reflecting the source and propagation of the activity. The scale bar in FIG. 4J is 100 ms. FIG. 4K shows an example of a current source density heat map of a sample nerve action potential from FIG. 4J as visualized across adjacent electrodes and reveals source localization and propagation. The scale bar in FIG. 4K is 5 ms.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

1. A method of preparing a composite, comprising
forming a dispersion of a conducting polymer;
evaporating the dispersion to form conductive sheets;
cutting the conductive sheets into fragments;
crushing the fragments to produce a particle suspension;
performing filtering on the particle suspension to produce filtered particles;
suspending the filtered particles to produce a filtered particle suspension;
sonicating the filtered particle suspension to produce a sonicated suspension;
precipitating the sonicated suspension to produce precipitated particles; and
combining the precipitated particles with an ion-conducting scaffolding polymer matrix to produce the composite.

2. The method of claim 1, wherein the dispersion includes crosslinkers.

3. The method of claim 1, wherein the conducting polymer is poly(3,4-ethylenedioxythiophene)-poly(styrenesulfonate) (PEDOT:PSS).

4. The method of 3, further comprising forming the conducting polymer by mixing 80% PEDOT:PSS, 20% ethylene glycol, and 0.6% 4-dodecyl benzene sulfonic acid (DBSA).

5. The method of claim 1, wherein the conductive sheets have a thickness of less than 50 μm.

6. The method of claim 1, wherein evaporating the dispersion comprises dehydrating a mixture PEDOT:PSS and 1% v/v of 3-glycidoxypropyltrimethoxysilane (GOPS).

7. The method of claim 1, wherein the mixture is dehydrated for at least eight hours at 120° C.

8. The method of claim 1, wherein the fragments are crushed in a bead mill.

9. The method of claim 8, wherein the bead mill has beads with diameters of 3.17-6.35 mm.

10. The method of claim 1, wherein the fragments are suspended in Iso Propyl Alcohol (IPA) while being crushed.

11. The method of claim 1, wherein filtering comprises filtering the particle suspension using a first filter having pores having a first size to produce first-filtered particles and then filtering the first-filtered particles using a second filter having pores having a second size to produce second-filtered particles, wherein the first size is less than the second size.

12. The method of claim 11, wherein the first size is 10 μm and the second size is 20 μm.

13. The method of claim 1, wherein sonicating the filtered particle suspension is performed at 40 kHz for at least eight hours.

14. The method of claim 1, further comprising forming the ion-conducting scaffolding polymer matrix, wherein forming the ion-conducting scaffolding polymer matrix comprises dissolving chitosan (CS)-based polymers in 2% v/v acetic acid, filtered using 40 μm cell strainers, and then concentrated via dehydration to 2.5% w/v to produce a chitosan stock.

15. The method of claim 14, wherein forming the ion-conducting scaffolding polymer matrix further comprises preparing a 40% w/v D-sorbitol solution using the chitosan stock.

16. The method of claim 15, wherein forming the ion-conducting scaffolding polymer matrix further comprises adding PEDOT particles into the sorbitol solution to form a PEDOT particle suspension.

17. The method of claim 16, wherein adding PEDOT particles into the sorbitol solution comprises adding PEDOT particles into the sorbitol solution while stirring the sorbitol solution to produce a 2.5:1 PEDOT:CS by weight suspension.

18. The method of claim 16, wherein forming the ion-conducting scaffolding polymer matrix further comprises dehydrating the PEDOT particle suspension to 0.1% w/v of PEDOT in a final solution.
